(19) United States
(12) Patent Application Publication
(10) Pub. No.: US 2025/0228752 A1
(43) Pub. Date: Jul. 17, 2025

(54) SOLID CARE AND/OR MAKEUP COMPOSITION

(71) Applicant: L V M H RECHERCHE, SAINT JEAN DE BRAYE (FR)

(72) Inventors: Emilie GOMBART, SAINT JEAN DE BRAYE (FR); Juliette BIDARD, SAINT JEAN DE BRAYE (FR)

(73) Assignee: L V M H RECHERCHE, SAINT JEAN DE BRAYE (FR)

(21) Appl. No.: 18/854,382

(22) PCT Filed: Apr. 5, 2023
(86) PCT No.: PCT/FR2023/050489

§ 371 Date: Oct. 4, 2024

(30) Foreign Application Priority Data
Apr. 5, 2022 (FR) FR2203098

(51) Int. Cl. A61K 8/02; A61K 8/27; A61Q 1/06; A61Q 1/10

(52) U.S. Cl. CPC A61K 8/0216; A61K 8/27; A61K 2800/651; A61Q 1/06; A61Q 1/10

(57) ABSTRACT

The present invention relates to a cosmetic composition in solid form, preferably anhydrous, for caring for and/or making up keratin materials, in particular the skin, eyelids, eyebrows or lips, comprising, in a physiologically acceptable medium: (I) zinc oxide particles, (ii) boron nitride particles, and (iii) one or more waxes.

### FIELD OF THE INVENTION

The present invention relates to the cosmetic field and in particular to solid cosmetic compositions for caring for and/or making up keratin materials, in particular the skin, eyelids, eyebrows or lips.

### STATE OF THE ART

Solid anhydrous skin or lip care and/or makeup products are sought after by consumers for their coverage and staying power properties.

Coverage is generally provided by white pigments and/or fillers, and titanium dioxide is generally used which has a strong opacifying and coverage power. But alternative solutions to titanium dioxide are increasingly sought and the Applicant has observed that zinc oxide particles, used to replace titanium dioxide in solid anhydrous compositions, tend to destructurize the solid composition and impact manufacturing feasibility as well as application and sensorial properties. This problem therefore requires the search for formulation solutions to maintain the structural properties of the stick and the application and sensorial properties of the stick, without impacting the coverage result provided by the zinc oxide particles. It is in particular sought to satisfy mechanical requirements on the one hand in order to ensure the feasibility of manufacturing, the holding of the bud during application and to prevent it from breaking and, on the other hand, the transfer qualities to guarantee comfortable application as well as sufficient deposit and good qualities on the lips (homogeneity of deposit and shade for example).

Unexpectedly, the Applicant observed that the use of boron nitride particles has allowed to resolve this problem and to obtain an anhydrous solid composition having good coverage properties, good mechanical properties of the stick and good application and sensorial properties.

### DISCLOSURE OF THE INVENTION

A first aspect of the invention is therefore a cosmetic composition in solid form, preferably anhydrous, for caring for and/or making up keratin materials, in particular the skin, eyelids, eyebrows or lips, comprising, in a physiologically acceptable medium:
(i) Zinc oxide particles,
(ii) Boron nitride particles, and
(iii) One or more waxes.

According to a particular and preferred embodiment, the composition of the invention is an anhydrous composition.

The present invention also relates to a cosmetic method for caring for and/or making up keratin materials, in particular the skin, the eyelids, the eyebrows or the lips, preferably the lips, comprising the application to said keratin materials of a composition as defined in the invention.

### DETAILED DESCRIPTION OF THE INVENTION

The present invention therefore relates to a cosmetic composition in solid form, preferably anhydrous, for caring for and/or making up keratin materials, in particular the skin, eyelids, eyebrows or lips, comprising, in a physiologically acceptable medium:
(i) Zinc oxide particles,
(ii) Boron nitride particles, and
(iii) One or more waxes.

“Anhydrous composition” means in particular that water is preferably not deliberately added to the composition of the invention but may be present in trace amounts in the various compounds used in the composition. In particular, the composition according to the invention comprises less than 4% by weight of water, preferably less than 3%, preferably less than 2%, more preferably less than 1%, even more preferably less than 0.5% by weight of water, relative to the total weight of said composition, or even is completely free of water. According to a particular embodiment, the composition is free of water.

“Composition in solid form”, according to the invention means a composition having, at a temperature of 20° C. and at atmospheric pressure (760 mm Hg), a hardness greater than 30 Nm⁻¹, preferably greater than 40 Nm⁻¹.

The hardness can be measured at 20° C. by the method called “butter-cutting wire” method, which consists in cutting a stick of product transversely, which is preferably cylindrical of revolution, using a rigid metal wire of diameter 250 μm by moving the wire relative to the stick at a speed of 60 mm/min. The hardness of the samples is expressed in Gram force (Gf) and can be measured using a texturometer such as TAXT Plus Texturometer.

For each type of product, a reference range (target) is established to guarantee the mechanical properties of the stick.

The solid composition according to the invention will generally have a hardness greater than or equal to 70 g, in particular greater than or equal to 90 g, for example ranging from 100 g to 300 g, and preferably ranging from 140 g to 210 g. Such compositions (corresponding in particular to sticks cast in standard format of approximately 12.7 mm), have adequate stability and hardness: the stick is sufficiently rigid and solid, does not break during application and is therefore compatible with packaging in a bud or in another solid form and with application by friction on the surface to be treated and/or to be made up; they are also characterized by ease of application such as good gliding, good disintegration upon application and a comfortable texture.

The composition may in particular be in the form of a stick or a composition packaged in a cup.

The composition of the invention comprises zinc oxide particles.

### Zinc Oxide Particles

Zinc oxide particles appear as a white powder and are insoluble in water.

Zinc oxide particles can be in a heterogeneous form, generally hexagonal and cubic.

The zinc oxide particles used according to the invention are pigmentary zinc oxide particles, as opposed to the zinc oxide nanoparticles used as a mineral sunscreen.

The average size of the zinc oxide particles used according to the invention will generally range from 0.1 to 50 μm (micrometers), in particular from 0.1 to 10 μm, or even from 0.1 to 1 μm.

The particle size can be measured by laser diffraction distribution method with a Malvern Mastersizer type apparatus from Malvern, in particular by measuring the value D[50], which represents the maximum size represented by 50% by volume of the particles.

Mention can in particular be made of the commercial references RONACARE ZINC OXIDE from the company Merck, or the reference A120-ZnO-11S3 from the company Kobo, or the reference ZINC OXIDE COSMETICS from the company Keyser&Mackay/Grillo.

According to a particular and preferred embodiment, the zinc oxide particles contain more than 80% by weight of zinc oxide (ZnO), in particular more than 95%, 96%, 97%, 98%, or 99% by weight of ZnO, or even 100% ZnO.

The zinc oxide particles used according to the invention may or may not be treated with a lipophilic treatment agent for better dispersion in the fatty phase.

According to a particular embodiment of the invention, the zinc oxide particles are not treated and they have the INCI name: Zinc oxide.

According to another particular embodiment, the zinc oxide particles are treated with a lipophilic treatment agent.

The zinc oxide particles can also be mixed with a wetting agent to facilitate their dispersion in the composition and thus improve the homogeneity of the composition.

According to a particular embodiment, the content of zinc oxide particles in the composition of the invention will generally range from 0.1% to 50% by weight of zinc oxide, in particular from 0.5 to 40% by weight, in particular from 1% to 30% by weight of zinc oxide, preferably from 2% to 20% by weight of zinc oxide relative to the total weight of the composition.

For an anhydrous solid composition for the skin and distinct from a powder (for example complexion corrector), the total content of zinc oxide particles will generally range from 0.5% to 30%, better from 1% to 20% by weight (of zinc oxide) relative to the total weight of the composition.

For an anhydrous solid composition for the lips (for example stick for the lips), the total content of zinc oxide particles will generally range from 0.1% to 30%, preferably from 0.5% to 20% (of zinc oxide) by weight relative to the total weight of the composition.

According to a particular and preferred embodiment of the invention, the composition comprises less than 3% by weight of titanium dioxide relative to the total weight of the composition, in particular less than 1%, or is even free of titanium dioxide.

According to an even more preferred embodiment, the composition of the invention is free of titanium dioxide.

The composition of the invention also comprises boron nitride particles.

### Boron Nitride Particles

The particles of boron nitride particles which can be used according to the invention are generally of lamellar or platelet shape. ‘Lamellar particle’ or ‘platelet particle’ means that the thickness of the particle is less than the width of the particle, in particular the ratio between the width and the thickness of the particle ranges from 2 to 100.

Boron nitride particles generally have the INCI name: Boron nitride.

The boron nitride particles which can be used according to the invention generally have an average size ranging from 0.5 to 20 μm, in particular from 1 to 10 μm, measured according to a laser diffraction distribution method described previously.

Mention can in particular be made of the following commercial references: BORON NITRIDE CC and SOFTOUCH CC from the company SAFIC ALCAN/Momentive, BORON NITRIDE SHP from the company IWASE/Mizushima, RONAFLAIR from Merck, CARESS BN from Kobo.

The particles of boron nitride particles may or may not be treated with a lipophilic treatment agent.

According to a particular embodiment, the boron nitride particles are not treated.

According to a particular embodiment, the boron nitride particles are not in the form of composite particles.

According to a particular embodiment, the content of boron nitride particles in the composition of the invention will range in particular from 0.1% to 50%, in particular from 0.2% to 40% by weight, preferably from 0.5% to 30% by weight, and better still from 1% to 20% by weight relative to the total weight of the composition.

According to a particular and preferred embodiment of the invention, the weight ratio between the zinc oxide particles and the boron nitride particles will range from 99:1 to 50:50, preferably from 99:1 to 80:20, preferably from 99:1 to 90:10.

The fatty phase of the composition further comprises one or more waxes.

### Waxes

“Wax”, within the meaning of the present invention, means a solid compound at 25° C. which has a reversible solid/liquid state change and a melting temperature greater than 30° C., preferably greater than 45° C.

Mention can be made of microcrystalline waxes, paraffin waxes, polyethylene waxes, ozokerite, products comprising a mixture of polyethylene and alcohols containing 20 to 50 carbon atoms, silicone waxes in particular alkyl dimethicones, C20-C40 alkyl stearates, beeswax, waxes of plant origin, copolymers of maleic anhydride and alpha-olefin, waxes obtained by metallocene catalysis, and mixtures thereof.

According to a particular and preferred embodiment, the total content of wax(es) in the composition of the invention ranges from 3% to 25%, preferably from 4 to 20% by weight, in particular from 5 to 15% by weight relative to the total weight of the composition.

The fatty phase will generally also comprise one or more oils.

“Oil” means, within the meaning of the invention, a fatty substance, not soluble in water, liquid at 25° C. and atmospheric pressure.

As oils which can be used in the invention, mention may in particular be made of hydrocarbon oils, silicone oils and mixtures thereof. According to a particular embodiment, the composition of the invention comprises one or more hydrocarbon oils.

As hydrocarbon oils, mention may in particular be made of hydrocarbon oils of vegetable or mineral origin, linear or branched C8-C19 alkanes, synthetic C₁₀-C₄₀ ethers, synthetic C₁₀-C₄₀ esters, C₁₂-C₂₆ fatty alcohols optionally branched and/or unsaturated, higher C₁₂-C₂₂ fatty acids optionally branched and/or unsaturated, mono- and/or polyesters of fatty acids and/or fatty alcohols and mixtures thereof.

As an example of oils of vegetable origin, mention is made of soybean oil, grape seed oil, sesame oil, corn oil, rapeseed oil, sunflower oil, cottonseed oil, avocado oil, olive oil, castor seed oil, jojoba seed oil, peanut oil, sweet almond oil, palm oil, pumpkin oil, hazelnut oil, apricot kernel oil, wheat germ oil, calophyllum oil, sesame oil, macadamia oil, coconut oil, cereal germ oil; candlenut oil, thistle oil, candellila oil, safflower oil, shea butter, and mixtures thereof.

As an example of a C10-C26 aliphatic fatty alcohol, mention is made of linear or branched, saturated, C10-C26, more preferably C16-C20 such as 2-octyldodecanol, aliphatic monoalcohols.

As an example of linear or branched hydrocarbons, mention is made of paraffin oils and their derivatives, petroleum jelly, mineral oils (mixtures of hydrocarbon oils derived from petroleum), polybutene, hydrogenated polyisobutene, polyisoprene, polydecenes such as hydrogenated polydecene, or else among linear, branched and/or cyclic alkanes.

As an example of possibly branched and/or unsaturated fatty acids, mention is made of myristic acid, palmitic acid, stearic acid, behenic acid, oleic acid, linoleic acid, linolenic acid or isostearic acid, and mixtures thereof.

As an example of esters, mention is made of monoesters or polyesters of fatty acids whose linear or branched fatty chain contains from 6 to 30 carbon atoms such as those mentioned above, or mixtures thereof, and fatty alcohols whose fatty chain contains from 3 to 30 carbon atoms, and in particular mono-and polyesters of hydroxy acids and fatty alcohols, esters of benzoic acid and fatty alcohols, polyesters of polyols, dipentaerythrityl C5-C9 esters, or polyglycerol polyesters, trimethylolpropane polyesters, propylene glycol polyesters, hydrogenated castor oil polyesters, and more particularly isononyl isononanoate, stearyl octanoate, isopropyl palmitate, isopropyl myristate, butyl stearate, hexyl laurate, 2-ethylhexyl palmitate, 2-hexyldecyl laurate, 2octyldecyl palmitate, 2-octyldodecyl myristate, 2-diethylhexyl succinate, diisostearyl malate, 2-octyl-dodecyl lactate, glycerin triisostearate or diglycerin triisostearate; diisostearyl malate, octyldodecyl stearyl stearate, cetearyl isononanoate, diisopropyl adipate, caprylic capric triglyceride, glycerol tricaprocaprylate, isocetyl stearoyl stearate, alkyl benzoate, (di) pentaerythrityl, pentaerythrityl tetraisostearate, dipentaerythrityl pentaisononanoate, bis-diglyceryl polyacyladipate-2, trimethylolpropane triethylhexanoate, triisostearate trimethylolpropane, propylene glycol dibenzoate, propylene glycol dioctanoate, mixtures of triglycerides obtained by esterification of glycerol with capric acid and caprylic acid, non-oxyalkylenated polyglycerol esters such as polyglyceryl-2 triisostearate, polyglyceryl-2 isostearate or polyglyceryl-2 diisostearate, isostearyl lactate, octyldodecyl hydroxystearate, diisostearyl malate, glycerin stearate; diethyleneglycol diisononanoate; jojoba esters, i polyglyceryl polyricinoleates such as polyglyceryl-3 polyricinoleates, polyglyceryl-5 polyricinoleates, polyglyceryl-6 polyricinoleates, and polyglyceryl-10 polyricinoleates (in their INCI names), and mixtures thereof.

The oil content can range from 10 to 90%, preferably from 20 to 60% by weight relative to the total weight of the composition.

According to a particular embodiment, the composition of the invention further comprises one or more pasty fatty substances.

“Pasty fatty substance” means a non-crystalline fatty compound comprising, at a temperature of 25° C., a liquid fraction and a solid fraction.

According to the invention, the pasty fatty substance can be selected from the group consisting of lanolin and its derivatives, polymeric silicone compounds, alkyl (meth) acrylate copolymers preferably having a C8-C30 alkyl group, homo and copolymer oligomers of vinyl esters having C8-C30 alkyl groups, homo and copolymer oligomers of vinyl ethers having C8-C30 alkyl groups, fat-soluble polyethers resulting from polyetherification between one or more C2-050 diols, ethylene oxide and/or propylene oxide copolymers with long chain C6-C30 alkylene oxides, diglycerol esters, arachidyl propionate, phytosterol esters, non-crosslinked polyesters resulting from the polycondensation between a diacid or a linear or branched C4-C50 carboxylic polyacid and a diol or a polyol, the ester resulting from the esterification reaction of hydrogenated castor oil with isostearic acid such as a monodi- or tri-isostearate of hydrogenated castor oil, a mixture of soy sterols and oxyethylenated (5 OE) oxypropylenated (5 OP) pentaerythritol, triglycerides of fatty acids and their derivatives, shea butter, cocoa butter, mango oil or butter, and mixtures thereof.

Mention may in particular be made of triglycerides of fatty acids and their derivatives, esters of polyol(s) and of fatty acid diacid dimer, or of one of its esters, esters of hydrogenated castor oil, oligomeric esters of glycerol, in particular diglycerol esters, vegetable butters and mixtures thereof.

The content of pasty fatty substances in the composition of the invention can range from 0 to 30% by weight, in particular from 5 to 15% by weight relative to the total weight of the composition.

### Additional Ingredients

The composition of the invention may further comprise an ingredient selected from lipophilic gelling agents, additional fillers, additional coloring materials and mixtures thereof.

According to a particular embodiment, the composition further comprises at least one lipophilic gelling agent.

### Lipophilic Gelling Agents

“Lipophilic gelling agent” according to the invention means a compound capable of gelling the fatty phase of a composition.

The lipophilic gelling agent can advantageously be selected from natural or synthetic clays; modified natural micas such as aluminum, magnesium and potassium fluorosilicate; dextrin and fatty acid esters such as dextrin palmitate or dextrin myristate; tri-esters of C8 C30 fatty acid and of mono- or poly-glyceryl such as glyceryl tri(hydroxystearate) (INCI name: Trihydroxystearin).

The lipophilic gelling agent is preferably a natural or synthetic clay selected from bentonites, in particular hectorites and montmorillonites, beidellites, saponites, nontronites, sepiolites, biotites, attapulgites, vermiculites and zeolites. Preferably, it can be selected from hectorites. Even more preferably a hectorite modified with a quaternary alkylammonium chloride is used, said ammonium being substituted by at least one, and preferably at least two C14-C20 alkyl radicals, such as disteardimonium hectorite in which the ammonium comprises two methyls and two stearyls.

As a preferred fat-soluble gelling agent, mention may be made in particular of a modified hectorite dispersed in an oil, for example the reference BENTONE GEL from Elementis with the INCI name Octyldodecanol (and) Disteardimonium Hectorite (and) Propylene Carbonate.

Thus, according to a particular embodiment, the composition of the invention further comprises a lipophilic gelling agent, in particular a mineral gelling agent such as bentones, preferably a quaternary hectorite.

When the composition comprises one or more gelling agent(s), the total content of lipophilic gelling agent(s) may range from 0.5 to 5%, in particular from 2 to 4% by weight relative to the total weight of the composition.

According to a particular embodiment, the composition further comprises at least one additional filler, distinct from zinc oxide particles and boron nitride particles.

### Additional Fillers

The additional fillers serve in particular to modify the rheology or texture of the composition and/or to confer a mattifying and/or blurring effect.

For the purposes of the invention, “fillers” means particles of any shape, colorless or white, of mineral or organic nature, natural or synthetic, which are in a form (platelet, spherical or oblong), insoluble and dispersed in the middle of the composition. The fillers are selected in particular from micas, of natural or synthetic origin; talc; bismuth oxychloride; silica;

kaolin; calcium carbonate, magnesium carbonate and hydrocarbonate; zinc, magnesium or lithium stearate, zinc laurate, magnesium myristate; powders of synthetic polymers, such as polyethylene, polyesters, polyamides (for example nylon); polyacrylic or polymethacrylic acid powders, silicone powders; cellulose powders; glass and ceramic beads; powders of organic materials of natural origin such as corn, wheat, rice or cotton starches, crosslinked or not; hydroxyapatite and mixtures thereof.

According to a particular embodiment, the composition of the invention does not comprise talc.

The fillers may or may not be treated on the surface, in particular by a lipophilic or hydrophobic agent; such as those described previously for zinc oxide particles.

According to a particular embodiment, the filler is not treated on the surface.

According to another particular embodiment, the filler is treated on the surface with a lipophilic agent such as those described above.

According to a particular embodiment of the invention, the composition comprises one or more additional coloring materials (distinct from zinc oxide particles).

### Additional Coloring Materials

The term “coloring material” within the meaning of the present invention means a compound capable of producing a colored optical effect when formulated in sufficient amount in an appropriate cosmetic medium. The coloring materials are conventionally selected from pigments (insoluble in oils and in the aqueous phase) and colorants (soluble in oils or in the aqueous phase).

Coloring materials can be of mineral, synthetic or natural origin.

According to a particular embodiment, the coloring materials are of mineral or synthetic origin.

Said coloring materials may or may not be treated with a lipophilic treatment agent.

A coloring material can be selected from organic or inorganic coloring materials, materials with an optical effect, and mixtures thereof, soluble or insoluble in the fatty or oily phase of the invention.

According to a particular embodiment, the coloring material(s) are selected in particular from mineral pigments distinct from zinc oxides, colorants and organic pigments, composite pigments, nacres, colorants, and mixtures thereof.

“Pigments” mean white or colored, mineral or organic particles, which are insoluble in an aqueous solution, intended to color and/or opacify the resulting deposit. Mineral pigments, organic pigments, and composite pigments (that is to say pigments based on mineral and/or organic materials) can be mentioned.

Among the “mineral pigments” distinct from zinc oxides, mention can be made of iron oxides, in particular black, yellow, red and brown iron oxides; manganese violet; ultramarine blue, chromium oxide, hydrated chromium oxide and ferric blue. As indicated above, according to a particular embodiment, the composition of the invention comprises less than 3% by weight of titanium dioxide relative to the total weight of the composition, in particular less than 1%, or is even free of titanium dioxide.

Preferably, the composition will comprise one or more iron oxides.

Among the “colorants and organic pigments (for example: lacquers)”, mention can be made, for example, of Blue 1, Yellow 5, Yellow 6, Yellow 10, Yellow 11, Red 4, Red 6, Red 7, Red 9, Red 19, Red 13, Red 17, Red 21, Red 22, Red 27, Red 28, Red 30 and Red 33, Red 34, Red 36, Red 40, Green 3, Orange 4, Violet 2, Carmine, Green 5, Orange 5, Orange 10, Green 6, Sudan red, Sudan brown, Orange 5, and quinoline yellow, their salts, such as a sodium or barium salt, or else lacquers comprising at least one of said colorants or organic pigments, for example those with the name Blue 1 lake (CI 42090); Yellow 5 lake (CI 19140); Yellow 6 lake (CI 15985), Yellow 10 lake (CI 47005), Yellow 11 Lake, Red 6 lake (15850); Red 7 lake (CI 15850); Red 21 lake (CI 45380); Red 22 lake (CI 45380), Red 27 lake (CI 45410), Red 28 lake (CI 45410), Red 30 lake (CI 73360), Red 33 lake (CI 15880), Red 34 lake (CI 15880), Red 40 Lake (CI16035), and/or their sodium or barium salts, in particular Red 6 Na Salt Lake, Red 6 Ba Salt Lake, and mixtures thereof.

“Lacquers” are organic pigments comprising at least one organic colorant precipitated on an inert support (otherwise called “base”). In particular, lacquers comprising a base selected from alumina (Al), barium sulfate (Ba), or calcium rosinate (Ca) bases will be used. For example Red 28 Al lake, is a lacquer comprising an alumina base on which the Red 28 colorant is precipitated.

According to a more particular embodiment, the composition comprises at least one colorant or organic pigment (for example lacquer) selected from the group consisting of Blue 1, Blue 1 lake (CI 42090), Red 6, Red 6 lake (15850), Red 7, Red 7 lake (CI 15850), Red 27, Red 27 lake (CI 45410), Red 28, Red 28 lake (CI 45410), and/or their sodium or barium salts, and mixtures thereof; preferably Red 27, Red 27 lake (CI 45410), Red 28, Red 28 lake (CI 45410), and/or their sodium or barium salts, and mixtures thereof; more preferably Red 28, Red 28 lake (CI 45410), and/or their sodium or barium salts, and mixtures thereof.

By “nacres”, mention can in particular be made of colored pearlescent pigments, such as mica with iron oxides and with or without TiO2, mica with an organic pigment of the aforementioned type, as well as pigments based on bismuth oxychloride. Mention can be made of the commercial reference nacre ranges Reflecks®, Ronastar®, Timiron® and Syncristal®. Pearlescent pigments may also have a base of silica, alumina, alumina hydroxide, synthetic mica, tin oxide, borosilicate (for example: calcium sodium borosilicate, calcium aluminum borosilicate or cellulose or cellulose derivatives).

According to a particular embodiment, the composition of the invention comprises one or more additional coloring materials selected from the group consisting of iron oxides, colorants or organic pigments, nacres, and mixtures thereof.

According to a particular embodiment, the composition comprises an additional coloring material selected from the group consisting of:
(i) Colorants and organic pigments selected from the group consisting of Blue 1, Blue 1 lake (CI 42090), Yellow 5, Yellow 5 lake (CI 19140), Yellow 6, Yellow 6 lake (CI 15985), Yellow 10, Yellow 10 lake (CI 47005), Yellow 11, Yellow 11 lake, Red 4, Red 4 lake (CI 14700), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), Red 30, Red 30 lake (CI 73360), Red 33, Red 33 lake (CI 15880), Red 34, Red 34 lake (CI 15880), Red 40, Red 40 Lake (CI 16035), Orange 4, carmine, and/or their salts, in particular Sodium or Barium salts, such as Red 6 Na Salt Lake, Red 6 Ba Salt Lake, and mixtures thereof; in particular Blue 1, Blue 1 lake (CI 42090), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), and/or their salts, and mixtures thereof; preferably Red 27, Red 27 lake (CI 45410), Red 28, Red 28 lake (CI 45410), and/or their salts, and mixtures thereof;
(ii) Composite pigments comprising an inorganic and/or organic substrate, in particular a silica, a borosilicate or an alumina, in which at least one of the aforementioned colorants or organic pigments are embedded or encapsulated, and mixtures thereof;
(iii) Pearlescent pigments comprising a substrate, in particular mica, covered with at least one of said colorants or organic pigments mentioned above, and mixtures thereof; in particular pearlescent pigments comprising a synthetic mica covered with a colorant or organic pigment selected from Blue 1, Blue 1 lake (CI 42090), Yellow 5, Yellow 5 lake (CI 19140), Red 6, Red 6 lake (15850), Red 7, Red 7 lake (CI 15850), Red 27, Red 27 lake (CI 45410), Red 28, Red 28 lake (CI 45410), carmine, and mixtures thereof;
(iv) and mixtures thereof;
more preferably the colorants or organic pigments selected from Red 27, Red 27 lake (CI 45410), Red 28, and Red 28 lake (CI 45410), or a composite pigment comprising Red 27, Red 27 lake (CI 45410), Red 28, or Red 28 lake (CI 45410), or a nacre comprising Red 27, Red 27 lake (CI 45410), Red 28, or Red 28 lake (CI 45410), and mixtures thereof.

In particular, the coloring material(s) are present in the composition in a total content (including the content of zinc oxide particles) ranging from 0.1% to 25% by weight, relative to the total weight of the composition.

For a care composition, the total content of coloring materials (including the content of zinc oxide particles) will generally range from 0.1 to 5% by weight relative to the total weight of the composition.

For a makeup composition, the total content of coloring materials (including the content of zinc oxide particles) will generally range from 5 to 25% by weight, in particular from 8 to 15% by weight relative to the total weight of the composition.

According to a particular embodiment, the composition of the invention comprises:
Zinc oxide particles,
Boron nitride particles,
One or more waxes in a total content ranging from 3 to 25% by weight relative to the total weight of the composition,
Hydrocarbon oils,
Optionally a lipophilic gelling agent,
Optionally one or more coloring materials selected from the group consisting of iron oxides, colorants and organic pigments, nacres, and mixtures thereof,
and characterized in that it comprises less than 3% by weight of titanium dioxide relative to the total weight of the composition, in particular less than 1% by weight of titanium dioxide, or is even free of titanium dioxide.

According to a particular and preferred embodiment, the composition of the invention comprises:
Zinc oxide particles, in particular in a content of 0.5 to 40% by weight relative to the total weight of the composition,
Boron nitride particles, in particular in a content of 0.2 to 40% by weight relative to the total weight of the composition,
One or more waxes in a total content ranging from 3 to 25% by weight relative to the total weight of the composition,
Hydrocarbon oils,
One or more lipophilic gelling agents,
One or more coloring materials selected from the group consisting of iron oxides, colorants and organic pigments, nacres, and mixtures thereof,
and characterized in that it is free of titanium dioxide.

According to a preferred embodiment, the weight ratio between the zinc oxide particles and the boron nitride particles in these preferred embodiments of compositions of the invention ranges from 99:1 to 50:50, in particular from 99:1 to 80:20, preferably 99:1 to 90:10.

### Galenic

The composition of the invention is a composition for caring for and/or making up keratin materials, in particular a composition for the skin, eyelids, eyebrows, or lips, preferably eyelids, eyebrows and lips.

According to a particular embodiment, the composition of the invention is in the form of a stick or of a composition packaged in a pot or a cup.

Depending on a particular and preferred embodiment, it will be a stick for the lips or a composition in a pot or cup for the lips.

According to another particular embodiment, it will be a pencil liner.

According to another particular embodiment, it will be a stick or a pencil or a composition in a pot or cup, applicable with the finger or with a brush, for the eyebrows (for example: eyebrow pencil).

According to another embodiment, it will be a stick or a composition in a pot or cup, applicable with the finger, with a sponge or with a brush, for the eyelids (for example: eyeshadow).

According to another embodiment, it will be a stick or a composition in a pot or cup, applicable with the finger, with a sponge or with a brush, for the skin of the face, in particular certain areas (for example: complexion corrector or concealer).

Thus, the composition of the invention is in particular in the form of a stick for the lips, a liner pencil, an eyebrow pencil, a blush or eyeshadow, or a complexion corrector or concealer, preferably a stick for the lips.

### Cosmetic Method

The invention also relates to a cosmetic method for caring for and/or making up keratin materials comprising the application, to said keratin materials, in particular the skin, the eyelids, the eyebrows, or the lips, preferably the lips, of a composition according to the invention.

The compositions of the invention are as defined above.

According to a particular embodiment, the composition of the invention applied to keratin materials comprises:
Zinc oxide particles,
Boron nitride particles,
One or more waxes in a total content ranging from 3 to 25% by weight relative to the total weight of the composition,
Hydrocarbon oils,
Optionally a lipophilic gelling agent,
Optionally one or more coloring materials selected from the group consisting of iron oxides, colorants and organic pigments, nacres, and mixtures thereof,
and characterized in that it comprises less than 3% by weight of titanium dioxide relative to the total weight of the composition, in particular less than 1% by weight of titanium dioxide, or is even free of titanium dioxide.

According to a particular and preferred embodiment, the composition of the invention applied to keratin materials comprises:
Zinc oxide particles, in particular in a content of 0.5 to 40% by weight relative to the total weight of the composition,
Boron nitride particles, in particular in a content of 0.2 to 40% by weight relative to the total weight of the composition,
One or more waxes in a total content ranging from 3 to 25% by weight relative to the total weight of the composition,
Hydrocarbon oils,
One or more lipophilic gelling agents,
One or more coloring materials selected from the group consisting of iron oxides, colorants and organic pigments, nacres, and mixtures thereof,
and characterized in that it is free of titanium dioxide.

Preferably, the composition applied to the skin or the lips is in the form of a stick for the lips, a liner pencil, an eyebrow pencil, a blush or eyeshadow, or a complexion corrector or concealer, preferably a stick for the lips.

The invention will now be illustrated in the following non-limiting examples. The % are expressed as % by weight of ingredient relative to the total weight of the composition, unless otherwise indicated.

### EXAMPLES

### Example 1: Effect of Boron Nitride Particles on the Structure and Application and Sensorial Properties of the Composition

Several tests were carried out upstream to determine the coverage power of zinc oxide particles, compared to titanium dioxide, in order to be able to replace titanium dioxide in formulas, without impacting the desired coverage effect. These results showed that, for an equivalent coverage power, in an anhydrous solid composition according to the invention, an amount of zinc oxide particles 3 to 4 times greater than the amount of titanium dioxide was required for an equivalent coverage result.

In the following examples, a formula with 1.4% by weight of titanium dioxide (‘TiO2’) relative to the total weight of the composition was therefore used as a control formula.

The formula with particles of zinc oxide alone (‘ZnO’) replacing TiO2 comprises a content of 4.4% by weight of ZnO. But the addition of this amount of ZnO tends to destructure the stick, making the stick more difficult to apply, more crumbly and the deposit on the lips is less homogeneous.

The Applicant therefore sought solutions to compensate for these deleterious effects on the structure of the stick and the application and sensorial properties of the stick, and used boron nitride (‘BN’) particles.

The examples of the control (TiO2), comparative (ZnO alone) formulas and the formula according to the invention (ZnO+BN) are described in the following table.

TABLE 1

| | Control | Comparative | Invention |
|---|---|---|---|
| | (TiO2) | (ZnO alone) | (ZnO + BN) |
| INGREDIENTS | % by weight | % by weight | % by weight |
| WAXES (synthetic wax, | 15 | 15 | 15 |
| microcrystalline wax) | | | |
| FILLERS (other than boron nitride | 2 | 2 | 2 |
| particles) | | | |
| DISTEARDIMONIUM HECTORITE | 1 | 1 | 1 |
| PASTY FAT (cholesterol esters and | 6.5 | 6.5 | 6.5 |
| butter of vegetable origin) | | | |
| CALCIUM CARBONATE | 2 | 2 | 2 |
| YELLOW and BLUE LAKES and DC | 5.9 | 5.9 | 5.9 |
| RED 6 and RED 28 Lake | | | |
| TITANIUM DIOXIDE | 1.4 | — | — |
| ZINC OXIDE PARTICLES | — | 4.4* | 4.2 |
| BORON NITRIDE PARTICLES | — | — | 0.2 |
| CONSERVATIVES | Qs | Qs | Qs |
| OILS (esters and octyldodecanol) | Qsp 100 | Qsp 100 | Qsp 100 |

*the amount of ZnO is approximately 3 times greater than the amount of TiO2 to obtain the same coverage power and to be ‘iso-tinted’ in the examples.

### Protocol for Preparing the Compositions

The compositions are prepared according to the following procedure:

All the coloring particles (ZnO, TiO2 and organic pigments/lacquers) are prepared beforehand in the form of pigment paste: each pigment or lacquer is dispersed in an oily medium. A majority of the oils, pasty fatty substances, modified hectorite, preservatives and waxes are weighed and heated to 95° C. Stirring allows homogenization just after heating.

Then the pigment pastes are added. The mixture is passed under a rotor-stator for good homogenization in the medium.

The fillers are then added, everything being homogenized before casting at a temperature comprised between 85 and 90° C. The mold is left to rest at room temperature for 10 minutes before being placed in the freezer for 5 minutes.

The sticks are unmolded and left to rest at room temperature in their packaging (stick).

### Evaluation of the Properties of the Sticks Obtained

The structural properties of the stick (in particular its hardness), and the application and sensorial properties (in particular the homogeneity of the deposit and the gliding ability upon application, are evaluated.

The hardness properties of the different sticks are evaluated at 20° C. by the method called “butter-cutting wire” method, which consists in cutting a stick of product transversely (cylindrical of revolution), using a rigid metal wire with a diameter of 250 μm by moving the wire relative to the stick at a speed of 60 mm/min. The hardness of the samples was evaluated using a texturometer such as TAXT Texturometer and is expressed in Gram force (Gf). The value obtained for a lipstick is generally comprised between 70 and 300 g, knowing that, for the composition of the invention, a hardness as close as possible to the control example comprising the same composition but with TiO2 instead of ZnO (the hardness range here is between 140 and 190 g) is sought.

The results of the evaluations are illustrated in the following table:

TABLE 2

| Control | Comparative | Invention |
|---|---|---|
| (TiO2) | (ZnO alone) | (ZnO + BN) |
| The stick has a good gliding | The stick is more difficult to | The stick has a good |
| upon application, a | apply, it is more crumbly. | gliding upon application, |
| homogeneous deposit and has | The deposit on the lips is less | a homogeneous deposit |
| a rigid structure | homogeneous. | and a rigid structure, |
| | | comparable to the control |
| | | with TiO2 |

These results show that the use of boron nitride particles in a composition comprising zinc oxide particles as a replacement for titanium dioxide and in an amount sufficient to obtain an equivalent coverage power, allows to compensate for the unfavorable effects related to zinc oxide particles on the structure and application and sensorial properties of the stick, and thus restore the desired properties (comparable to the control with TiO2) in a composition without TiO2.

### Example 2: Formulations

The following examples are illustrative and non-limiting examples of the invention, prepared according to methods well known to the cosmetic formulator.

### 2. 1 Anhydrous Stick for the Lips

TABLE 3

| INGREDIENTS | % |
|---|---|
| SILICA | 5.0 |
| POLYETHYLENE WAX | 5.5 |
| CANDELILLA WAX | 3.0 |
| SHEA BUTTER | 1.5 |
| IRON OXIDE (red) | 5.0 |
| ORGANIC PIGMENTS (LAQUERS) | 2.0 |
| BORON NITRIDE PARTICLES | 0.15 |
| ZINC OXIDE PARTICLES | 3.0 |
| HYDROGENED POLYDECENE | QSP100 |

The lipstick has the expected properties in terms of structure and sensoriality upon application. Applied to the lips, the lipstick provides good coverage and color properties with a homogeneous deposit.

### 2.2 Anhydrous Eyeshadow

TABLE 4

| Ingredients (INCI Name) | % by weight |
|---|---|
| SILICA | 15 |
| SYNTHETIC FLUORPHLOGOPITE | 10 |
| IRON OXIDE | 8 |
| ZINC OXIDE PARTICLES | 10 |
| BORON NITRIDE PARTICLES | 3 |
| NACRES | 2 |
| OILS AND WAXES | Qsp 100 |

The eyeshadow, packaged in a cup, has the expected properties in terms of structure and sensoriality upon application. Applied to the eyelids, the eyeshadow provides coverage and a pearly effect with a homogeneous deposit.

### 2.3 Anhydrous Stick for the Lips

TABLE 5

| INGREDIENTS | % |
|---|---|
| SILICA | 5.0 |
| POLYETHYLENE WAX | 5.5 |
| CANDELILLA WAX | 3.0 |
| SHEA BUTTER | 1.5 |
| IRON OXIDE (red) | 2.0 |
| ORGANIC PIGMENTS (LAQUERS) | 5.0 |
| BORON NITRIDE PARTICLES | 0.5 |
| ZINC OXIDE PARTICLES | 8.0 |
| HYDROGENED POLYDECENE | QSP100 |

The lipstick has the expected properties in terms of structure and sensoriality upon application. Applied to the lips, the lipstick provides good coverage and color properties with a homogeneous deposit.

1-11. (canceled)

12. A cosmetic composition in solid form, for caring for and/or making up keratin materials, comprising, in a physiologically acceptable medium:
(i) Zinc oxide particles
(ii) Boron nitride particles, and
(iii) One or more waxes,
characterized in that it comprises less than 3% by weight of titanium dioxide relative to the total weight of the composition.

13. The cosmetic composition according to claim 12, which is an anhydrous composition.

14. The cosmetic composition according to claim 12, wherein the total content of wax(es) ranges from 3 to 25% by weight relative to the total weight of the composition.

15. The cosmetic composition according to claim 12, wherein it further comprises one or more pasty fatty substances.

16. The cosmetic composition according to claim 12, wherein it comprises less than 1% of titanium dioxide relative to the total weight of the composition.

17. The cosmetic composition according to claim 16, which is free of titanium dioxide.

18. The cosmetic composition according to claim 12, wherein the content of zinc oxide particles ranges from 0.1 to 50% by weight, relative to the total weight of the composition.

19. The cosmetic composition according to claim 12, wherein the content of boron nitride particles ranges from 0.1 to 50% by weight, relative to the total weight of the composition.

20. The cosmetic composition according to claim 12, wherein the weight ratio between the zinc oxide particles and the boron nitride particles ranges from 99:1 to 50:50.

21. The cosmetic composition according to claim 12, wherein it further comprises another additional ingredient selected from lipophilic gelling agents, additional fillers, additional coloring materials and mixtures thereof.

22. The cosmetic composition according to claim 12, which is in the form of a stick or a composition packaged in a pot or a cup.

23. The cosmetic composition according to claim 22, which is a stick for the lips, a liner pencil, an eyebrow pencil, a blush or eyeshadow, or a complexion corrector or concealer.

24. The cosmetic according to claim 23, which is a stick for the lips.

25. A cosmetic method for caring for and/or making up keratin materials, comprising the application to said keratin materials of a composition as defined in claim 24.

26. The cosmetic method according to claim 25, wherein the composition is applied onto the skin, eyelids, eyebrows or lips.
